(19) United States
(12) Patent Application Publication
(10) Pub. No.: US 2014/0012061 A1
(43) Pub. Date: Jan. 9, 2014

(54) NON-INVASIVE LOCATION AND TRACKING OF TUMORS AND OTHER TISSUES FOR RADIATION THERAPY

(71) Applicants: Xubo Song, Portland, OR (US); Martin Fuss, Portland, OR (US); Deniz Erdogmus, Portland, OR (US); Daryl Kaurin, Portland, OR (US)

(72) Inventors: Xubo Song, Portland, OR (US); Martin Fuss, Portland, OR (US); Deniz Erdogmus, Portland, OR (US); Daryl Kaurin, Portland, OR (US)

(73) Assignee: Oregon Health & Science University, Portland, OR (US)

(21) Appl. No.: 13/907,417

(22) Filed: May 31, 2013

Related U.S. Application Data
(62) Division of application No. 12/679,730, filed on Mar. 24, 2010, now abandoned.
(60) Provisional application No. 60/974,670, filed on Sep. 24, 2007.

(51) Int. Cl. A61N 5/10

(52) U.S. Cl. CPC A61N 5/103
USPC 600/1

(57) ABSTRACT

Embodiments herein provide a non-invasive tracking system that accurately predicts the location of tumors, such as lung tumors, in real time, while allowing patients to breathe naturally. This is accomplished by using Electrical Impedance Tomography (EIT), in conjunction with spirometry, strain gauge and infrared sensors, and by using sophisticated patient-specific mathematical models that incorporate the dynamics of tumor motion. With the direction and speed of lung tumor movement successfully tracked, radiation may be effectively delivered to the lung tumor and not to the surrounding healthy tissue, thus increased radiation dosage may be directed to improving local tumor control without compromising functional parenchyma.

### CROSS REFERENCE TO RELATED APPLICATIONS

The present application is a divisional of U.S. patent application Ser. No. 12/679,730, filed Mar. 24, 2010 entitled “Non-Invasive Location and Tracking of Tumors and Other Tissues for Radiation Therapy,” which is a U.S. National Phase of PCT/US2008/077445, filed Sep. 24, 2008 entitled “Non-Invasive Location and Tracking of Tumors and Other Tissues for Radiation Therapy,” which claims priority to U.S. Provisional Patent Application No. 60/974,670, filed Sep. 24, 2007, entitled “Non-Invasive Location and Tracking of Tumors and Other Tissues for Radiation Therapy,” the entire disclosures of which are hereby incorporated by reference in their entirety.

### TECHNICAL FIELD

Embodiments relate to the field of medical therapeutics, more specifically, to non-invasive location and tracking of tumors and other tissues to improve the effectiveness of radiation therapy.

### BACKGROUND

Current radiation therapy protocols for tumors, such as lung tumors, call for delivering highly concentrated dosages to the tumors within very tight volume and distribution margins. Determining the appropriate radiation dosage and the positioning of the tumor remain a challenge. With respect to a lung tumor, the tumor and body surface generally move during the treatment due to respiratory motion. This may significantly affect the precision of targeting the tumor and delivering radiation, during a single or multiple treatment sessions. Therefore, methods to effectively manage motion, such as respiratory motion, in radiation therapy are of substantial clinical importance.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example and not by way of limitation in the figures of the accompanying drawings.

FIG. 1 illustrates the internal anatomy of the lungs with Electrical Impedance Tomography (EIT) electrode array layers overlaid in accordance with various embodiments;

FIG. 2 illustrates an EIT 4-layer electrode array on a human subject in accordance with various embodiments;

FIG. 3 illustrates an EIT scanner schematic in accordance with various embodiments;

FIG. 4 illustrates a sequence of cross-sectional images of a chest obtained by EIT for a healthy subject, showing incremental (˜600 ml) inflation of the lungs starting from residual lung volume in accordance with various embodiments; and

FIG. 5 is an exemplary tumor tracking process flowchart in accordance with various embodiments.

### DETAILED DESCRIPTION OF DISCLOSED EMBODIMENTS

In the following detailed description, reference is made to the accompanying drawings which form a part hereof wherein like numerals designate like parts throughout, and in which is shown by way of illustration embodiments which may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments herein; however, the order of description should not be construed to imply that these operations are order dependent.

The description may use perspective-based descriptions such as up/down, back/front, and top/bottom. Such descriptions are merely used to facilitate the discussion and are not intended to restrict the application of embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

A phrase in the form “NB” or in the form “A and/or B” means “(A), (B), or (A and B)”. A phrase in the form “at least one of A, B, and C” means “(A), (B), (C), (A and B), (A and C), (B and C), or (A, B and C)”. A phrase in the form “(A)B” means “(B) or (AB)” that is, A is an optional element.

The description may use the phrases “in an embodiment,” or “in embodiments,” which may each refer to one or more of the same or different embodiments. Furthermore, the terms “comprising,” “including,” “having,” and the like, as used with respect to embodiments, are synonymous.

Embodiments herein enable effective and accurate management of motion, such as respiratory motion, for radiation therapy of tumors in the lungs, pancreas, liver, etc. Embodiments provide methods that identify the location of and track the motion of tissues such as tumors, for example lung tumors, non-invasively and in real time, while allowing patients to breathe naturally.

Embodiments may utilize Electrical Impedance Tomography (EIT) alone or in conjunction with various anatomical constraints and/or a suite of other external sensors (such as spirometry, infrared sensors, strain gauge, and/or body surface photogrammetry) and may use sophisticated patient-specific mathematical models that incorporate the dynamics of tumor motion.

Even though EIT provides a practical and effective modality for imaging lung ventilation compared to other existing imaging modalities, there is always innate ambiguity associated with tumor motion when using stand-alone imaging. Such ambiguity arises not only due to image imperfections, but also due to variations in the motion patterns of tumors, hysteresis and the asymmetry of tumor trajectories during inhalation and exhalation. In an embodiment, such underlying uncertainty may be resolved by modeling the dynamics of breathing motion, which takes into account the temporal characteristics of the tumor motion by considering the mechanical properties and the elasticity of the lung tissue. In an embodiment in which the direction and speed of lung tumors are successfully tracked, radiation may be precisely targeted to the lung tumor and not to the surrounding healthy tissue, and thus increased radiation dosage may be directed to improving local tumor control without destroying healthy tissues.

Thus, an embodiment provides a noninvasive tracking system that accurately predicts the location of tumors, such as lung tumors, in real time, while allowing patients to breathe naturally. In an embodiment, location and tracking may be accomplished by using one or more components of accurate tumor location and tracking: 1) sophisticated patient-specific mathematical models that incorporate the dynamics of tumor motion based on the mechanical and elastic properties of lung tissue; 2) Electrical Impedance Tomography (EIT); 3) anatomical constraints derived from MRI/CT/US, etc.; 4) external surrogate measurements from multiple sensors such as spirometers, reflective markers, and/or strain gauges; 5) multicamera photogrammetry to track surface electrode position; and/or 6) optimal sequential estimation of tumor location using Bayesian principles based on the dynamic model and noninvasive sensor measurements.

In an embodiment, once accurate tracking of tumor position and velocity has been achieved, a radiation beam may be controlled in real-time to deliver an increased dose more precisely to the tumor, improving local tumor control without compromising functional parenchyma, thus sparing healthy tissue and reducing treatment time while allowing the patient to breathe comfortably.

In some situations, EIT may not reveal the tumor location per se. However, according to an embodiment, EIT is intended to serve primarily as a monitor of the respiration, not as a direct monitor of the tumor location. The actual tumor location may be inferred from the respiration information contained in EIT, and the trained patient-specific breathing dynamic model, as well as the anatomical constraints derived from MRI/CT/US.

In addition, in an embodiment, EIT may be used to identify the location of the tumor (image guidance). The use of EIT as an image-guided intervention device provides a novel concept that may replace ionizing-radiation based image guidance, and may locate and/or track a tumor (rather than just a surrogate) directly, and may provide both inter-fraction and real-time intra-fraction tumor positional assessment.

In embodiments, software and/or hardware may be constructed to provide a finite element model (FEM), or another suitable model, such as a fused deposition model or a boundary element model, of EIT images.

In an embodiment, software and/or hardware may be provided for EIT-based image-guidance such that a tumor may be visualized and localized with a spatial accuracy, such as equal to or better than 5 mm. In an embodiment, to overcome limitations of current EIT imaging, and to increase useful image resolution, EIT hardware may be arranged with various channel arrangements, such as 32 measurement channels, 128 channels, or more to provide even greater spatial resolution. In an embodiment, image reconstruction may be based on 3D formulations, use of a single current source with parallel voltage measurement and the assumption of constant internal conductivities. In an embodiment, a forward model may use an anatomically constrained fixed finite element method (FEM) mesh developed from a 3D-CT scan of a suitable phantom. Impedances may be solved for using a typical nonlinear optimization approach.

In further embodiments, the tumor position is determined with or without the creation of associated images. In an embodiment, based on 4D-CT object motion data, a forward model may be based on a dynamic, parameterized FEM mesh, as well as predefined absolute tissue impedances derived from absolute EIT supplemented by diffusion tensor imaging (DTI). In embodiments, tracking methods may be enhanced by employing linearization to the computations and by use of techniques like Kalman filtering to follow and predict changes in target position. Patient-Specific Tumor Dynamics: In an embodiment, an accurate model of breathing motion under quiet respiration may be desirable to obtain accurate identification of tumor location. In other attempts, breathing motion has been modeled as a function of the breathing phase. One attractive aspect of a phase-based description of breathing is that many objects in the lung do not move along the same path during inhalation and exhalation due to hysteresis. Although the phase-based description is robust for a programmed mechanical phantom and regular breathing cycles, this description does not precisely characterize tumor motion during quiet, uncoached respiration, which is irregular in amplitude. Thus, in an embodiment, it may be beneficial for the motion of lung and lung tumor tissues to be modeled as a function that includes these five degrees of freedom, namely: (1-3) the three-dimensional position of the tissues, (4) tidal volume and (5) airflow, defined as the time derivative of the tidal volume. Since quiet respiration is not perfectly regular, the tidal volume may be defined based on a percentile system.

Hysteresis is generally caused by pressure disequilibria throughout the lung during breathing, which is in turn caused by differential airflow. Thus, the effects of hysteresis may be characterized as a function of airflow. A side benefit of this representation is that tidal volume may be conveniently measured using a spirometer, and airflow may be easily derived as the temporal derivative. In general, Newtonian mechanics describe the dynamics of moving objects using position, velocity, and acceleration in the three dimensional spatial coordinate system. In an embodiment, a mathematical model may be utilized to characterize the dependence of breathing motion on these state variables. Other approaches have used a simple linear motion model utilizing a five-state system, which is generally inadequate to account for the nonlinear deformation of lung tissue. Such a motion model does not consider the temporal dependency (the dynamics) of the five degrees of freedom, which may be used to resolve the ambiguity of tumor location when seen only from external measurements and images. Embodiments employ dynamic models stemming from Newton's mechanics of motion, as well as the elastic properties of the lungs. Thus, an embodiment provides a more accurate mathematical representation of tumor movement due to breathing.

For the modeling phase, in an embodiment, the ground truth (gold standard) for tumor position may be obtained via 4D-CT imaging, while the patient breathes naturally. In an embodiment, a regular CT scanner may be used to obtain a reconstructed 4D-CT sequence such as by using a methodology similar to the one described by Low et al., see Low et al., A Method for the Reconstruction of Four-Dimensional Synchronized CT Scans Acquired During Free Breathing, Medical Physics, vol. 30, no. 6, pp. 1254-1263, 2003, the entire contents and disclosure of which is hereby incorporated by reference. In an embodiment, patient specific unknown parameters of the dynamic model may be identified using statistical model fitting techniques. Simultaneously, in an embodiment, external sensor measurements may be recorded and a forward measurement model for the external sensors given the tumor/lung state may be provided.

In an embodiment, such a procedure provides a generative model of sensor measurements from the tumor state. For example, let the tumor state vector x(t) at phase t be composed of the instantaneous tumor position, velocity, acceleration, tidal volume, and airflow. Also, let the instantaneous external sensor measurements (including, in an embodiment, information from the EIT images) be collected in vector form s(t) at the corresponding state. The generative model may be represented as a general nonlinear dynamical system of the form:

\dot{x}=f(x,θ_{f}) s=h(x,θₕ)+n (Formula 1),

where n represents measurement noise and the tumor acceleration profile during breathing may be parametrically modeled from the collected data with 0 denoting the parameters of the model describing the dynamics.

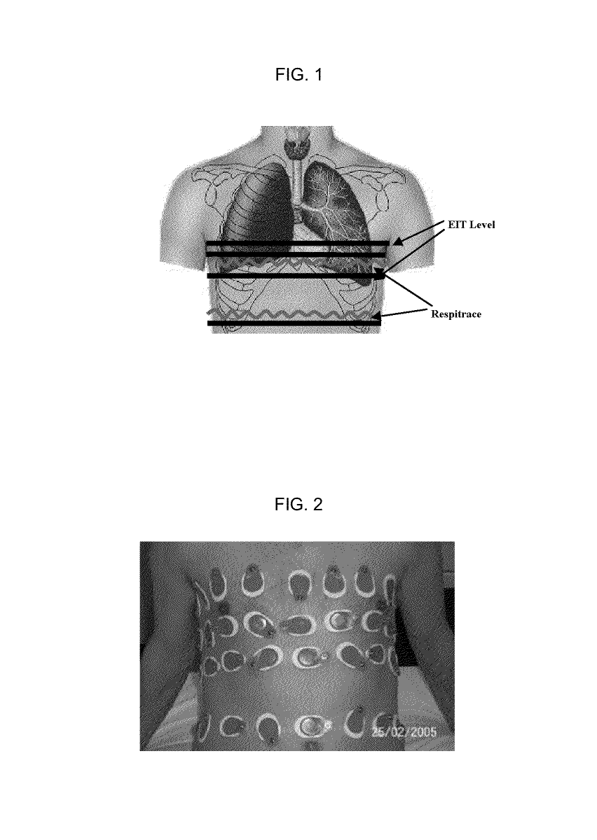

Electrical Impedance Tomography: EIT generates cross-sectional images of impedance distribution of the body through a set of electrodes placed in a transverse plane over an area of the body (see FIGS. 1 and 2). This is possible because the electrical resistivities of different body tissues vary widely (e.g., from 0.65 ohm/m for cerebrospinal fluid to 150 ohm/m for bone), so that an impedance distribution image may be formed.

To obtain a still image or video, a group of electrodes may be attached to a subject. The group consists of non-current-carrying and current-carrying electrodes. In an embodiment, the electrodes may be linked to a data acquisition unit that outputs the data, for example, to a PC or other computing device. In an embodiment, by applying a series of small currents to the current-carrying pairs of electrodes, a set of potential difference measurements may be made from non-current carrying pairs of electrodes. The electric currents applied to the body take the path of least impedance, where the currents' flow depends on the subject's conductivity distribution. In an embodiment, the image reconstruction process is a nonlinear optimization problem, for which there exist a variety of methods with which it may be solved. In an embodiment, data acquisition and image reconstruction may be performed in real time.

In an embodiment, an EIT system may be provided using, for example, 32 channels or 128 channels (or other numbers as desired). Such a system may use a single current source that may be switched electronically between any pair of electrodes. In an embodiment, the system may use parallel (simultaneous) measurement of the potential on the remaining (30 or 126, etc.) electrodes. In an embodiment, the system may use x-ray transparent electrodes and leads to allow for CT-scans without excessive artifacts.

In an embodiment, the system may use a multi-frequency digitally synthesized injection current, with maximal frequency of, for example, 10 KHz to 1 GHz. The use of multiple scanning frequencies provides a conductivity spectrum for each tissue type, and thus provides further conductivity contrast to differentiate tissues from each other, making the entire EIT process more robust. In addition, higher scanning frequencies allow faster data acquisition and thus higher temporal resolution in object position tracking. For a high degree of versatility, the system may be set to record the raw potentials at a high sample rate, for example at four to ten times the maximal scanning frequency, depending on the steepness of the anti-aliasing filters. Such a setting may allow visualization of artifacts and noise, and thus may provide opportunities to reduce or remove them. This is in contrast to most traditional EIT systems that perform sine wave amplitude extraction (demodulation) in hardware and thus never truly know what artifacts occurred and how they affect the data.

In an embodiment, several parameters of the system may be programmable, including the number and frequency of sine waves in the synthesized current injection waveform, the “dwell time” or switching speed between different injection pairs, the number and sequence of injection pairs used, and/or the current level. For absolute EIT imaging, all possible injection pairs may be used, but, in a dynamic tracking embodiment, the pairs may be limited to a smaller number determined from simulation and experiment. In embodiments, lower frequencies and more pairs both increase the dwell time and hence reduce the ability to track fast changes.

In an embodiment, current level may be limited to the maximal allowed leakage current for medical devices appropriate for the frequency of current injection used. The gain of the measurement amplifiers may be set appropriately to capture the full dynamic range of skin surface potentials expected to be encountered. An anti-aliasing filter may be used to prevent high-frequency noise from being digitized along with the signal. In an exemplary embodiment, a 24-bit analog-digital converter may be used and the sample rate of the converter (one per channel) may be set to match the roll-off of the filter. Phase shifts caused by the anti-aliasing filter may also be measured and factored into the analysis.

In accordance with a specific embodiment, given a high frequency of 10 KHz, 24-bit (3-byte) digitization, 32 channels, and a sample rate of 40,000 frames per second (KFps), there may be a data rate of about 4 MBytes per second (MBps); for a 128 channel system this increases to about 16 MBps. These are not unreasonable rates for streaming data to hard disk.

In an embodiment, there may be a battery powered front end in the hardware to ensure patient safety. After digitization and multiplexing of the signal from each electrode, the data may be serialized and sent over a fiber optic channel to an interface board in the controlling personal computer, which may, in an embodiment, be placed at a considerable distance from the subject and treatment equipment.

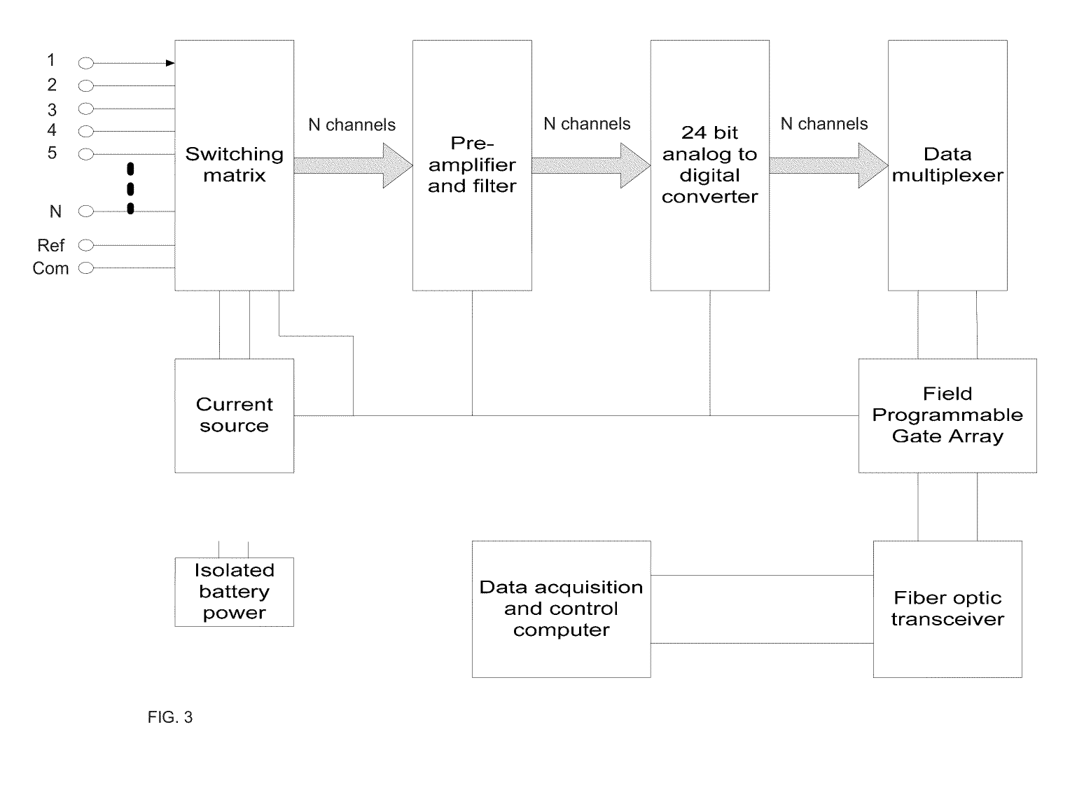

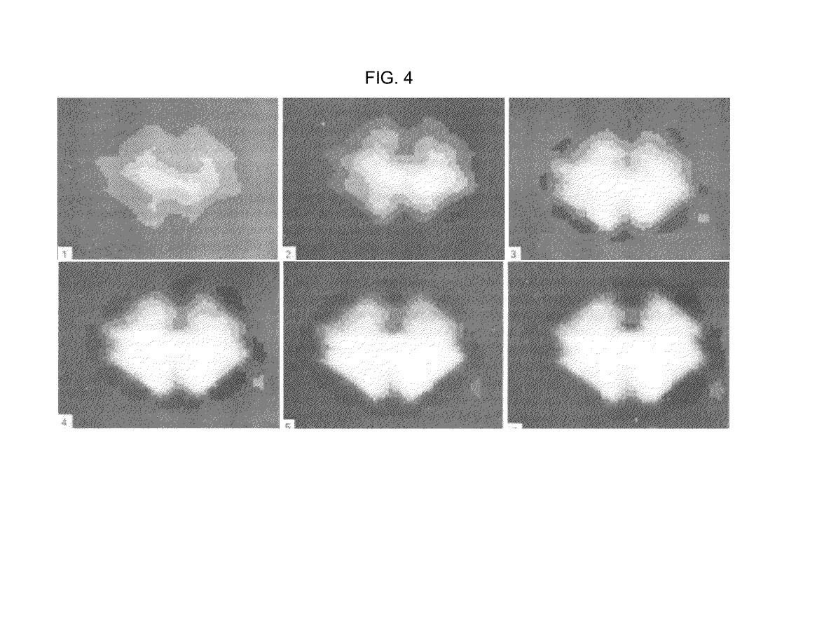

In accordance with embodiments, two exemplary architectures may be provided. Both approaches have a floating AC current source connected to skin electrode pairs via CMOS multiplexers. At any time, two electrodes may be driven with the current source and the remaining electrodes may be connected to low-noise preamplifiers to measure the voltage at each electrode. A digital controller (field programmable gate array (FPGA) or microprocessor) may sequence the multiplexers through all electrode combinations (see FIG. 3).

In one embodiment, a custom built electrode interface board that includes preamplifiers and 24 bit A/D (analog to digital) converters may be provided for each of the N electrode channels. The digital outputs from the A/Ds may be formatted by an FPGA on the board and formatted for transmission over a fiber optic link to a PC for data collection. The FPGA may also provide channel sequencing for the current injection. The interface board may be battery operated for safety isolation from ground.

In another embodiment, a custom built preamplifier board may be provided for the 32 channel electrode interface. In this embodiment, the output of the board is 32 analog signals. These voltages may be passed to a commercial 32 channel, 24 bit A/D board (such as General Standards 24DSI32) installed in a battery powered industrial PC chassis. The preamp board and PC may be isolated from ground. The digitized data may be stored on the PC disk and transmitted by a wireless link (WiFi or fiberoptic). Channel sequencing may be done with a simple FPGA or microcontroller on the preamplifier board.

Using various embodiments herein, EIT is suitable for imaging the lungs and ventilation in vivo, in part, since the lungs exhibit significant temporal electrical impedance changes as a result of respiration. In an embodiment, the relative impedance changes in the lungs, as assessed with EIT, may be proportional to changes in lung volume. In contrast with simple impedance pneumography, which provides global information on thoracic impedance, EIT offers the possibility of obtaining regional information on lung function with high specificity. As a result, in an embodiment, it is possible to study pulmonary functions under various physiological and pathological circumstances using EIT. Conductivity changes related to respiration may thus be imaged using EIT with excellent reproducibility. FIG. 4 shows snapshot EIT images of the lung during ventilation in a healthy subject (˜600 ml incremental lung volume).

In embodiments, EIT may provide image matrices on the order of 64×64, 128×128, or better. In an embodiment, an EIT-based image guidance system may provide for location of a target centroid with an accuracy of at least 5 mm, and may provide for tracking of the tumor, with 90-95% accuracy or better, for example, over assessment periods longer than 2 minutes.

In addition to the feasibility of EIT imaging biological objects under in vivo conditions in real time, its advantages over other imaging technologies in accordance with embodiments are in part that it provides a non-invasive and sensitive method to probe the body using nonionizing radiation, it may be operated by technicians with minimal training, it does not require patients to modify their breathing patterns, and it is suitable for long-term monitoring. Compared to many other imaging modalities, the cost of EIT equipment is low (only about $25,000). Furthermore, EIT generates data not provided by other imaging techniques, namely data about the electrical properties of tissue.

In an embodiment, EIT, as an external sensing/imaging technology, may be used to track the changing locations of tumors. Unlike other external sensors, such as strain gauges and infra-red markers that measure displacements of marker locations or chest expansion strains on the skin surface, EIT may be used in accordance with embodiments to identify and quantify changes in internal lung anatomy during respiration by constructing cross-sectional images of the electrical impedance distribution within the chest cavity and chest organs, including tumors in the lung. Such a tool increases tumor tracking accuracy by introducing novel information about the internal structures. In an embodiment, image slices (similar to a CT slice) obtained using EIT rings near the tumor allow for real-time registration of these images with pretreatment CT scans.

Anatomical Constraints: In addition to EIT measurements, one or more anatomical constraints may be provided to improve the spatial resolution and speed of EIT. Such constraints may be obtained from magnetic resonance imaging, computed tomography, ultrasound, etc.

In an embodiment, a simple example of applying anatomical constraints comes from attempts to measure the static impedance of head tissues in order to construct an accurate electrical model of the head for electroencephalogram (EEG) modeling. Traditional EIT would require a fairly uniform mesh throughout the head, with thousands of unknown impedances to estimate. If we assume that the basic geometry of the head may be derived from CT and/or MRI scans, an FEM mesh may be constructed to match the various constituent tissues of the head, such as gray and white matter, bone, skin, fat, CSF, etc. If we further assume that each of these tissues has the same impedance everywhere, then the number of unknowns may be significantly reduced, and estimated much more quickly and robustly. In an embodiment, such an example may be extended to the use of anatomical constraints for lung tumor location and tracking.

In an embodiment, to create an FEM mesh for a patient or phantom, a corresponding CT data-set may be manually segmented into regions of uniform impedance using commercial radiation therapy structure segmentation software. Each defined region may be assigned a unique impedance in the model. These regions may then be fed to a software program that creates the FEM mesh and assembles the solid tetrahedral elements of the phantom. At this point, an embodiment deviates from traditional EIT in that such an embodiment may enforce uniform impedance in the regions previously defined. Instead of having an unknown impedance in each element of the mesh (numbering, for example, in the 100s or 1000s), such an embodiment has only a small number of unknowns (such as less than 10), one for each unique material in the CT data set.

In an embodiment, an EIT system may utilize software to calculate the surface potentials given a particular current injection pair and a particular set of impedances (forward problem). In an embodiment, the software may perform adaptive mesh refinement with the matrix equations solved by optimal order multi-grid methods.

In an embodiment, the inverse problem solution estimates the unknown impedances given the known geometry, applied currents, and measured voltages on the surface. The inverse problem in traditional EIT has generally been linearized in order to make it easier and faster to solve, but this also leads to distortions and artifacts in the images. Embodiments herein formulate the inverse problem for absolute impedance imaging using the true and exact relationship between current, voltage, 3D geometry, and impedance, and solve it with appropriate non-linear optimization algorithms. In addition, in an embodiment, the use of anatomical constraints (in the form of a priori knowledge of the location and range of impedances in the object derived from CT/MRI scans of the specific body) may greatly reduce the number of unknowns and may allow for correct solutions in a reasonable time.

Specialized firmware and hardware for computations: The solution of non-linear inverse problems is computationally intensive. Various techniques are available utilizing electronic and computing hardware and firmware (programmable hardware) for speeding up most computations, including digital signal processing (DSP) chips, array processing chips, field programmable gate arrays (FPGA), and parallel processing computer arrays. In an embodiment, one or more of these techniques may be incorporated. A particular embodiment may use an FPGA for each EIT channel programmed as a custom signal processor to demodulate the amplitude of the scanning frequency (or multiple frequencies). In addition, an embodiment may use a cluster of identical computers configured as a parallel processor to calculate the FEM forward solution at each time frame.

In biological objects, there may be variations in impedance within a particular tissue or object. In order to account for this variability, but not revert entirely to the traditional EIT formulation, embodiments may replace fixed values of impedance in a region with a distribution, where the impedance may take on a small range of values, more likely in the center of the distribution, which may be Gaussian or, in an embodiment, something more problem specific if a priori knowledge exists about the empirical distribution.

Noninvasive External Surrogate Measurements: In addition to the EIT measurements, one or more external measurement devices may be used in accordance with embodiments to track tumor and/or respiratory movement: spirometers, strain gauges, and reflective markers. These sensing modalities provide physical information that complements the EIT imagery. Specifically, a spirometer provides information regarding the global volume and airflow behavior of the lungs; the strain gauges, placed on the upper thorax and near the abdomen may be useful for assessing the effects of diaphragm movements (a major source of superior-inferior motion) near the two extremes of the lungs; and the reflective markers, positioned on a grid around the chest wall provide distributed spatial information about the movements of the skeletal structures and the connected lung tissue, which in turn provide useful landmark information that may also be exploited for alignment of the patient's body coordinate frame (the coordinate system according to which the tumor location is estimated) and the radiation delivery equipment's coordinate frame.

Existing studies on reflective markers utilize few (two to four) markers, to infer tumor location solely based on the measurement of the positions of these markers. More sensors in an array may lead to more accurate estimation (assuming statistically independent contributions from each additional sensor). Thus, in an embodiment, a larger number of markers, such as at least about 10, 15, 20, 30, 40, etc. may placed over the chest wall to increase accuracy. In an embodiment, the markers may be observed by multiple cameras and computer vision algorithms may be utilized to track their trajectories.

Photogrammetry: In addition to EIT measurements, one or more cameras may be provided to monitor and record surface sensor/marker movement in real-time. The 3D location of each marker or grid point may be calculated with standard stereo-photogrammetric triangulation, and input to the tracking software for mesh modification. In an embodiment, an external marker visible in CT and by camera may be used if needed for a reference point. In an embodiment, multicamera photogrammetry may be utilized with EIT to track object position directly.

Electrode and fiducial location measurement from photogrammetry: In order to follow surface changes, surface marker movement may be recorded using four cameras. Electrodes may be marked with labels having a high-contrast pattern to aid in identification and localization by software. In addition, a second set of markers may be attached in a grid pattern over the entire torso. These markers may have a slightly different pattern on them and may be used to track the shape of the torso. Alternatively, a grid pattern may be optically projected. Either way, the grid aids in identifying corresponding points in multiple camera images. Prior to tracking, each marker may be identified in each of the four initial camera views. During tracking, the software may automatically find each marker's new position, which is fairly straightforward since markers generally move only a small amount between video frames. In embodiments, image patch correlation may be used for tracking purposes. Then, the 3D location of each marker or grid point may be calculated with standard stereo-photogrammetric triangulation, and input to the software for mesh modification. An external marker visible in CT and by camera may be used if needed for a reference point.

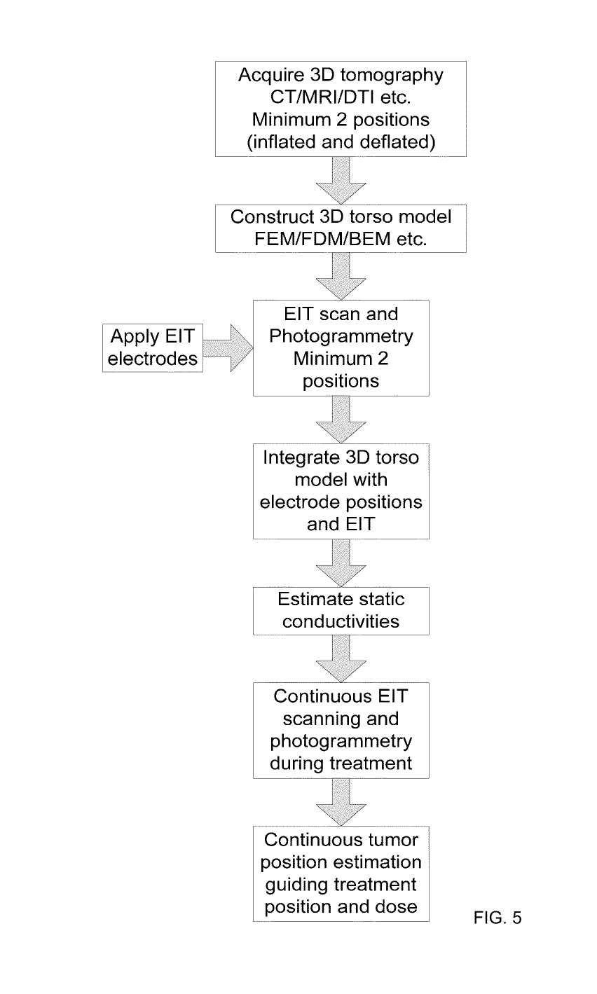

An exemplary high-level process flowchart in accordance with embodiments is presented in FIG. 5. As shown, a 3D tomography image is acquired at a minimum of two positions, chest inflated and chest deflated. A 3D torso model is constructed, for example using FEM, FDM, BEM, etc. EIT electrodes are applied separately to a body, and an EIT scan and photogrammetry are performed at a minimum of two positions, again inflated and deflated. The 3D torso model is integrated with the electrode positions and EIT. The static conductivities may be estimated. During treatment, continuous EIT scanning and photogrammetry may be performed providing continuous tumor position estimation guiding treatment position and dose.

In an embodiment, to address the difficulties of tracking a moving object, a further set of unknowns may be added to efficiently model the movement of tissue boundaries. In an embodiment, there may also be added additional measurements of the objects 3D external shape to partially account for these additional unknowns as well as the 4D-CT data which may be correlated with the external surface. In an embodiment, the problem may be constrained by assuming that the impedances of the various tissues and tumors are known. In an embodiment, one objective is to track the movement of boundaries, primarily the tumor itself, but secondarily all of the boundaries since they relate computationally. In embodiments, the unknowns include the set of control points of the FEM mesh, that is, a subset of the internal boundary nodes plus a subset of the external boundary nodes (electrode and marker or grid points). In an embodiment, the inverse problem is to track the control points of the tumor, which, assuming tumor rigidity, reduce to a center position vector and possibly an orientation vector.

Optimal Sequential State Estimation: In an embodiment, developments in recursive Bayesian tracking provide a framework and the mathematical formulation for estimating the current state of the tumor (including three-dimensional position and velocity of the tumor, the tidal volume, and the airflow), given multiple types of sensor measurements over time (EIT images, spirometer, strain gauge, and marker data). In an embodiment, such a formulation provides for the framing of the tumor tracking problem as an optimal state estimation problem.

State estimation is a general framework in statistical signal processing and dynamical system theory. Currently, extremely robust and accurate estimation algorithms exist for object tracking. Besides the classical Kalman Filter and its nonlinear extension the Extended Kalman Filter, Unscented Kalman Filters and Particle Filters have been developed that are extremely accurate in state estimation for nonlinear dynamical systems, the class of systems in which tumor dynamics fall. Thus, an embodiment adapts recursive Bayesian tracking to the study of tumor motion.

An embodiment provides a real-time estimation algorithm based on the mathematical model of tumor dynamics that continuously outputs estimated tumor location and velocity with corresponding confidence levels utilizing data from EIT images and other external sensors. Since the inverse estimation of tumor state from only measured data is ill-posed, the regularizing effect of the dynamical model may provide accuracy and consistency of the estimates. In an embodiment, the estimator is based on the classic representation of a discrete-time nonlinear dynamic system in state space (i.e., a discretized version of Formula 1):

xₖ₊₁=f(xₖ,vₖ;θ_{f})

yₖ₊₁=h(xₖ,nₖ;θₕ) (Formula 2),

where xₖ represents the unobserved (hidden) state of the system and yₖ is the only observed signal at time k. Process noise and observation noise are denoted by vₖ and nₖ, respectively. For the problem of tumor tracking, the state xₖ comprises tumor position, velocity, acceleration, as well as the tidal volume and airflow. The observation yₖ comprises data from the EIT images, the spirometer, and the other sensors. The function f, known as the state transition function, describes the dependency of current state on the previous state (i.e., if the tumor is at location A now, then it should be at location B next). In an embodiment, some level of uncertainty may be introduced by the process noise. The function h, known as the measurement mapping, describes how the current state determines the current observation (i.e., if the tumor is at location A now, then the images should look like these now). Functions f and h may be parameterized by corresponding vectors θ. The functional forms of f and h may be determined based on the mechanical and elastic properties of the lung tissue, or through model fitting. The unknown parameter θ may be determined in order to fully formulate the model. For this purpose, in an embodiment, patient specific parameter fitting may be performed using patient data that may include the location of the tumor, the EIT images and the other sensor measurements obtained in the modeling phase as described earlier. Once the functions f and h are known, in the treatment phase in accordance with an embodiment, the tumor location may be dynamically determined given the sensor measurements. This process is referred to as state estimation.

Thus, embodiments provide: 1) patient-specific models from data acquired from CT scans, EIT images, and other corresponding sensor data; 2) algorithms for specification of patient-specific mathematical models; this model specification process involves determining the parameter θ for each patient; and 3) algorithms for tumor location estimation using the patient-specific mathematical models, as well as EIT, spirometry, strain gauge, and marker monitoring data.

Data collection: In an exemplary embodiment, data may be collected from patients who have been diagnosed with peripheral lung tumors, although other tumors or tissues may be tracked as well. Each patient may be positioned on a CT scanner. In an embodiment, each patient may have eight electrodes (alternate numbers may be utilized) per layer/slice from the EIT machine attached to specific spots on their chests. In the meantime, in an embodiment, the patients may wear spirometry in their mouths, strain gauges on the thorax, and/or reflector markers on their chest walls. CT scans of a patient's tumor(s) may in an embodiment be taken while synchronously EIT images and other sensor measurements may be recorded. In an embodiment, all measurements may be taken using a 4D-CT over a period, such as a five-minute period, to cover multiple breathing cycles. Radiation exposure due to these CT scans is negligible compared to the dosage administered during treatment.

Since conventional CT images taken during free breathing may have artifacts due to breathing motion, in accordance with an embodiment, a 4D-CT may be reconstructed that more accurately depicts respiratory motion. In an embodiment, the 4D-CT involves monitoring periodic respiratory motion using spirometric data, acquiring image information at corresponding phases in the respiratory cycle using a multi-slice helical CT scanner, and reconstructing and collating all image information into image datasets, with each set representing a single phase in the respiratory cycle. The 4D-CT images provide multiple discrete, volumetric snapshots of the patient's lungs while breathing. In an embodiment, in order to detect the tumor from the 4D-CT images, image segmentation may be performed.

Specification of the state transition function: While some portions of the transition function are automatically given by Newtonian mechanics (e.g., position is the integral of velocity over time), the specification of certain parts of the patient-specific state transition model in accordance with an embodiment may be based on standard tools from function approximation and machine learning theory. In an embodiment, one technique that may be utilized is the temporal motion model of the moving thorax volume. This motion model characterizes non-rigid, free breathing with smooth lung motion using a weighted sum of shifted basis functions. Specifically (assuming that the complete transition function is approximated with this method for simplicity of notation

$${x}_{k + 1} = {x}_{k} + {\sum }_{r = 1}^{K} {\sum }_{i} {w}_{ri} b ( k / {Δ}_{t} - τ ) β ( {x}_{k} / {Δ}_{x} - i ) , \qquad ( Formula 3 )$$

where Δₓ controls the width of the spatial basis function β(·) and Δₜ controls the width of the temporal basis function b(·). The general approach applies to any differentiable basis function. In an embodiment, b(·) is a cubic B-spline, and β(·) is the tensor product of cubic B-splines. B-splines may be used for several reasons: they offer good approximation of band-limited signals and they may be used for modeling non-rigid deformation. The compact support of B-splines, and hence small overlap between knots, reduces the dependency between parameters, thus making the optimization problem easier to solve. Given this functional form of f, the parameters wᵣᵢ may be easily optimized using the experimental data minimizing a suitable error function, for example the sum of squared errors.

Specification of the measurement mapping: In an embodiment, measurement mapping describes what the observations (i.e., the EIT image and the spirometric data) should be given the tumor location. It is the projection of the internal unobservable state (i.e., tumor location) onto the sensor measurements. Such a projection reflects the lung anatomy during breathing and the EIT image formation property, as well as the spirometry and other external sensor characteristics. What functional form h assumes depends on how the EIT images are represented. The representation may be the whole EIT image, or it may comprise some salient features derived from the image, such as points, corners, contours or regions. Similarly for spirometry and other sensors, features of relevance may be included in the measurement formulation through probabilistic models. These features may provide a more compact and more relevant representation than the whole image, and they may be computationally more effective. In an embodiment, a feature extraction procedure may be used to detect these features. However, spurious features may be detected, hence the probabilistic modeling approach may be used. Embodiments provide a variety of image and signal representations, for example contours (represented by “snakes”), as well as the whole image for the EIT and wavelet based features as well as raw measurements for the other sensors.

Given any specific image representation, the functional form for h may be difficult to obtain physically due to the complexity of the EIT image formation process. One approach in accordance with an embodiment may be to assume a general nonlinear parameterized function for h, for instance an artificial neural network, and then fit the parameters by optimization. In an embodiment, this may however be difficult due to the high dimensionality of yₖ. In a further embodiment, a data-driven nonparametric approach for modeling h may be utilized. In an embodiment, the projection may be derived given a particular state using data from the corpus collected. In an embodiment, since the ground truth of the tumor state may be derived from the CT, and the simultaneously captured EIT images and sensor measurements may be obtained, optimal spline interpolation filters may be employed to the corpus of patient data as the ideal projection given a particular state. In an embodiment, this may be done for any image representation, and only requires a “look-up table” level of computational complexity.

Bayesian State Estimation: After the models are specified for a patient, in an embodiment, the tumor location may be dynamically estimated given sensor measurements. In an embodiment, tumor state estimation may be based on developments in recursive Bayesian tracking. With Bayesian inference, an estimate of the probability density of the system state xₖ (i.e., tumor location) given a sequence of observations (e.g., EIT images, spirometer and other sensor measurements) may be propagated.

The well-known Kalman Filter (KF) is a classical algorithm that implements optimal recursive Bayesian estimation in linear dynamical models with Gaussian noise. Its extension, the Extended Kalman Filter (EKF) has been utilized as an heuristic technique for nonlinear state estimation. Recent developments in state estimation rely on more accurate realizations of the Bayesian formulation in arbitrary nonlinear non-Gaussian dynamical models.

Using Bayes rule, the a posteriori conditional probability density of the state given all past observations may be recursively expressed as follows:

$$p ( {x}_{k} \vert {y}_{0 \text{:} k} ) = \frac{p ( {x}_{k} \vert {y}_{0 \text{:} k - 1} ) p ( {y}_{k} \vert {x}_{k} )}{p ( {y}_{k} \vert {y}_{0 \text{:} k - 1} )} . \qquad ( Formula 4 )$$

The first term in the numerator is the a priori estimate of the state distribution that is approximated, which may be expressed using the total probability theorem as

p(xₖ|y_{0:k-1})=∫p(xₖ|xₖ₋₁)p(xₖ₋₁1|y_{0:k-1})dxₖ₋₁ (Formula 5).

The second term in the numerator is simply the probabilistic measurement model determined by the measurement equation. Finally, the denominator in the posterior recursion, on the other hand, is the normalization term that is approximated conveniently in the practical algorithm through simple weight normalization.

This recursion specifies the current state density as a function of the previous density and the most recent measurement (observed) data. The lung kinematics and the dynamics of the tumor motion come into play through the state-transition probability p(xₖ|xₖ₋₁), which describes the likelihood of the current tumor location and velocity given a particular tumor state at the previous observation instant. The observation density p(yₖ|xₖ) represents the image and sensor measurement likelihoods given a particular tumor state, which describes the probability of observing a particular EIT image and sensor readings given the current tumor location. Once the dynamic equation (Formula 1) is specified, the state transition probability may be easily modeled. In an embodiment, a simple approach may be to assume additive noise, for instance Gaussian noise. Similarly, the likelihoods of the observation features (extracted from the EIT images and sensor signals) may be obtained utilizing the measurement equation of Formula 2.

After the state transition and observation probability density models have been specified, in an embodiment, an efficient propagation algorithm may be provided in order to carry out the recursive Bayesian formulation for state estimation. However, the multi-dimensional integration in Formula 5 makes a closed form solution intractable for most systems. In an embodiment, a workable approach may be to apply Monte-Carlo sampling techniques that essentially convert integrals to finite sums, which converge to the true solution in the limit (for large sample sizes). Under a pure Gaussian and linear assumption, the Kalman filter is optimal for the recursive propagation of all necessary terms. A first-order approximation to account for nonlinearities leads to the EKF, which is the current industry standard and the most widely used algorithm. The EKF, however, has certain theoretical and practical limitations, which often makes it difficult to implement and may even lead to filter divergence. In an embodiment, a Monte-Carlo sampling implementation of the Bayesian framework described above is the particle filter where the integral in Formula 5 is approximated by a sample average drawing a large random sample from the state transition probability distribution and utilizing importance sampling techniques for systems with intractably complex noise distribution models.

Particle filters are computationally expensive, requiring a large number of samples (particles) for reasonable accuracy. Thus, in an embodiment, a more efficient probabilistic framework may be used: Sigma-point Kalman Filters (SPKF). SPKF methods are a recent development in machine learning, and are shown to be far superior than EKF based estimation approaches. In an embodiment, SPKF filters may also be combined with particle filters for efficient Monte-Carlo simulations accounting for non-Gaussian distributions. These hybrid filters are referred to as Sigma-Point Particle Filters (SPPF).

Metrics for the Assessment of Performance: Currently available commercial systems deliver radiation in two modes: traditional isocentric beams that require the target tumor to be within the center of spherically distributed radiation sources and the modern (evolving) robotic arm delivery systems that offer 6-degrees-of-freedom (DOF) in delivering radiation from arbitrary locations and directions. While the traditional system is less flexible in delivery locations and relies on gating, it offers higher radiation doses per unit time, potentially decreasing the total treatment time. Modern systems relying on flexible robotic arm technology, however, do not support large accelerators, thus resulting in longer treatment times. In embodiments, two performance metrics may be used for the two types of accuracy definitions imposed by the available delivery mechanisms: root mean squared (RMS) distance error and gating error.

Simply increasing the delivered dose rate without improving accuracy may not be suitable for the purposes of certain embodiments. The RMS distance error, defined as the square root of the average tracking error between the estimated tumor coordinates and the center of mass of the tumor (in cm), may serve as an appropriate measure of performance that correlates well with the ratio of dose delivered to the tumor to the total dose delivered. While more accurate measures to assess the efficiency of radiation delivery by estimating the doses delivered to the tumor and the surrounding healthy tissue may be devised, current image processing technology may not be sufficiently reliable to assess this efficiency measure accurately in real time. In an embodiment, the RMS tracking error may be calculated for each patient separately over the whole duration of the testing phase data. The RMS errors of each patient may be normalized by the variance of the respective tumor trajectories in order to reduce the effect of patient differences. The average normalized RMS (NRMS) error may be utilized as the final performance metric.

Although it is likely that future delivery systems will employ flexible delivery mechanisms increasingly, to date these commercial systems are quite expensive and still evolving. Therefore, medical centers that have already invested in the traditional isocentric systems are unlikely to make the migration quickly. For such systems, the RMS error is not as useful in determining dose delivery efficiency. Since these systems rely on the delivery of radiation to the tumor when the tumor is within the region that may be targeted, an embodiment introduces the concept of gating error. The gating error metric quantifies the error in the control signal that determines the gating (on/off) decisions. In an embodiment, the gating system activates the radiation delivery when the tumor is in the target area and turns off the beam when it is not.

Two kinds of gating errors are possible: false positives and false negatives. False positives are the instances where the tumor is outside the target region, but the beam is turned on. False negatives are the instances where the tumor is inside the target region, but the beam is turned off. A higher risk may be associated with false positives, since radiation of healthy tissue poses a greater threat to the patient than missing a suitable delivery window that results in longer treatment time. Since the actual risk assignments are generally determined by the clinician for the specific patient, at this stage, in accordance with an embodiment, the receiver operating characteristics (ROC) may be compared by plotting the curves of false positive versus false negative probabilities for different thresholds of the detector that makes the decision. The ROC curves are a standard metric for evaluating binary hypothesis testing accuracy in probabilistic environments where actual Bayesian risk assignments cannot be made with high certainty. In an embodiment, the ground truth for the decision may be determined as follows: 1) a sphere with radius rₜᵤₘₒᵣ that encloses the tumor completely at a fully exhaled lung state may be determined by the clinician from the CT images; 2) a region with r_{target} (r_{target}>rₜᵤₘₒᵣ) that is either co-centric with this sphere or that is slightly displaced in the direction of expected tumor movement diverging from this state during inhalation may be denoted as the target region; and 3) the tumor may be considered to be within the target region if 50% of the sphere that encloses the moving tumor is within the target region.

Clearly, the larger r_{target} is the more likely it is for the tumor to be within the target region (i.e. shorter treatment time and increased damage to healthy tissue). In an embodiment, the margin may be determined in the treatment-planning phase by the clinician considering the medical condition of the patient, as well as the desired duration of treatment (typically 10-20 minutes). At any rate, given the ground truth, one may easily determine the false negatives and false positives: (i) the probability of a false positive is the ratio of the duration where the estimated tumor position is falsely in the target region to the total treatment duration; and (ii) the probability of a false negative is the ratio of the duration where the estimated tumor position is falsely outside the target region to the total treatment duration.

Embodiments herein have advantages over other related technologies. Current techniques that target lung tumors have severe limitations. For example, the breath-holding technique during irradiation minimizes tumor motion by controlling patients' breathing actively or passively; however, not all patients are good candidates for this technique since their impaired lung function does not allow them to repeatedly hold their breath for an extended period of time that is needed for treatment (usually 15-30 sec are needed for each hold). Respiratory gating radiation therapy is a technology that synchronizes the exposure of the radiation beam to part of the respiratory cycle when tumor motion is least. This method still results in a significant amount of residual tumor motion. In addition, both the respiratory gating radiation therapy and the breath-holding technique deliver radiation only during a short segment of the breathing cycle; the duty cycle, defined as the ratio between the particular portion of the breathing cycle when radiation is delivered and the entire breathing cycle, is typically 20-50%, so treatment time necessarily increases in order to deliver the prescribed dosage. An abdominal compression technique employs a stereotactic body frame with a flexible plate that presses against the abdomen during radiation treatment, but still permits limited normal respiration. This technique has met with success in minimizing diaphragmatic excursions and in reducing body movement, but causes discomfort for patients and only minimally reduces respiratory motion. Adaptive radiotherapy technology involves the continuous re-alignment of the radiation field so that the radiation beam follows the moving tumor. This technology uses either internal fiducials or non-invasive, external surrogates. With internal fiducials, gold marker seeds (2-mm diameter gold spheres) are implanted in or near the tumor using either a percutaneous or bronchoscopic implanting technique. The location of the tumor is determined during treatment by detecting the gold markers using standard X-ray technology. With external surrogates, sensors are placed externally on the patients, for instance on the surface of the chest, with the hope that their positions and measurements will serve as surrogates to reflect the internal lung ventilation or tumor movement. Typical surrogate sensors include infra-red reflective markers, strain gauges, spirometry, and video tracking. The adaptive radiotherapy technique has the advantage of being able to deliver treatment continuously throughout the radiation treatment. However, implanting internal gold markers requires skilled hands, is risky, and has led to serious complications (e.g., pneumothorax) in many patients. This technique may also adversely affect tumor localization if swelling occurs from marker implantation.

Of the current technologies and techniques, external surrogates provide a promising, non-invasive approach for tracking tumor motion in real time. However, the surrogates that are currently used in certain situations (infra-red reflective markers, strain gauges, spirometry, video tracking, fluororoscopy) are generally not sufficient, alone or in combination, to determine the precise location of a moving lung tumor. This is because the surrogates are only indirectly related to tumor movement; the true tumor motion cannot be unambiguously observed and uniquely determined through these surrogates. For instance, while the skin surface may move in the vertical direction, the diaphragm, which drives the lung motion, may internally move in the horizontal direction at the same time.

Actual imaging of lung tumors is a more direct way to locate them. However, current imaging modalities are not practical for this purpose, especially when a patient needs to be imaged for the entire period when radiation therapy is delivered. For instance, MRI is expensive and cumbersome. The image quality may degrade due to motion artifacts. Imaging the patient with CT for the whole treatment duration exposes the patient to high doses of radiation. Fluoroscopy-based images may not clearly visualize lung ventilation, and it provides misleading estimates of the actual tumor location.

Identifying the location of moving tumors is further complicated by the motion patterns, which vary considerably across patients, and by the trajectory of a tumor, which takes a different path during inhalation than it does during exhalation, a phenomenon known as hysteresis. Further, tumors move along the trajectory at different speeds during inhalation and exhalation. Differing speeds and trajectories of moving tumors necessarily complicate identifying their location at any particular moment during respiration, suggesting that more sophisticated techniques and technologies may be needed to successfully track moving lung tumors for radiation treatment.

Embodiments thus provide a non-invasive tracking system that may accurately predict the location of tumors, such as lung tumors, in real time, while allowing patients to breathe naturally. This may be accomplished by using EIT, in conjunction with spirometry, strain gauge and infrared sensors, and by using sophisticated patient-specific mathematical models that incorporate the dynamics of tumor motion. With the direction and speed of lung tumor movement successfully tracked, radiation may be effectively delivered to the lung tumor and not to the surrounding healthy tissue, thus increased radiation dosage may be directed to improving local tumor control without compromising functional parenchyma.

Although certain embodiments have been illustrated and described herein for purposes of description of the preferred embodiment, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A method of delivering radiation to tissue in a body, comprising:
locating and/or tracking movement of the tissue in the body by obtaining one or more images of the body or a portion thereof using electrical impedance tomography, and analyzing the one or more images obtained using electrical impedance tomography to locate and/or track the tissue movement; and
delivering radiation to the tissue in real time during tissue movement.
